(19) United States
(12) Patent Application Publication
(10) Pub. No.: US 2013/0005022 A1
(43) Pub. Date: Jan. 3, 2013

(54) PHOTO-BIOREACTOR AND METHOD FOR CULTIVATING BIOMASS BY PHOTOSYNTHESES

(76) Inventor: Peter James MORRIS, Nr. Dartmouth (GB)

(21) Appl. No.: 13/546,545

(22) Filed: Jul. 11, 2012

(30) Foreign Application Priority Data
Jan. 14, 2010 (GB) 1000593.2
Jan. 14, 2011 (GB) PCT/GB2011/000046

(51) Int. Cl. C12M 1/42; C12N 1/12

(52) U.S. Cl. 435/257.1; 435/292.1

(57) ABSTRACT

A method of cultivating biomass by photosynthesis. The method includes providing a fluid growth medium in which biomass is dispersed and which light can penetrate, exposing the growth medium to a source of incident light characterised in that the incident light is lensed using lensing means so as to form one or more elongate light foci in the growth medium, and transporting growth medium through said foci in a direction transverse or oblique with respect to a longitudinal foci axis so as to provide temporal photo-modulation in the growth medium.

### CROSS-REFERENCE TO RELATED APPLICATION

This application is a continuation of PCT Patent Application Serial No. PCT/GB2011/000046, filed Jan. 14, 2011, which claims the benefit of Great Britain Patent Application 1000593.2 filed on Jan. 14, 2010, which is hereby incorporated herein by reference.

### TECHNICAL FIELD

The present invention relates to the cultivation of biomass, such as phytoplankton/microalgae/cyanobacteria. The invention relates to the field of photo-bioreactors (PBR) and in particular those used for growing algae using solar light. The invention also relates to improved methods of growing algae.

### BACKGROUND

There has been great interest recently in third generation biofuels derived from alga, with the promise of huge potential yields. Cultivation vessels may be grouped into two general classes; those that are open to the environment, such as simple ponds, and those which have controlled atmospheres so as to permit control of reactor conditions and to prevent contamination of the biomass. The latter type may be termed “closed” or photobioreactors PBR. The simplicity and low-cost of production of open systems is let down by poor efficiency, whereas the potential high efficiency of closed PBR systems do not make up for the expense (energy, material and financial) of construction and operation.

A photo-bioreactor is essentially a “greenhouse” or more accurately an “aquarium” for cultivating alga/cyanobacteria in a water-based growth medium. It can be referred to as “test tube algaculture” as it is possible to control most parameters under near-laboratory conditions. Under the right conditions, alga can attain high rates of photosynthesis, enabling them to reproduce very rapidly and compile storage products resulting in large biomass yields.

The nature and properties of the harvested product depends on the type, strain and species of alga, growth conditions, and can take the form of, in decreasing value: pharmaceutical products, food, animal feed and the feedstock for a variety of biofuels (methane, diesel, hydrogen). There is potential to grow biomass in large-scale PBRs on marginal land in any coastal region to replace fossil fuels. Open ponds are currently used to culture algae due to prohibitive PBR construction costs. Open systems do however have many problems, not least competition from extraneous organisms in the growth medium, temperature regulation and evaporative water loss.

A typical photosynthesis (PS) efficiency prediction (according to J. Benemann, Algen Stammtisch, Hamburg, Oct. 9, 2008) includes the following factors:
˜90% of light reaches the algae,
˜45% of this is photosynthetic active radiation (PAR),
˜90% of photons are absorbed by photosynthesis pigments,
˜22% maximum photosynthesis efficiency (i.e. photons→biomass energy)

However there are further losses, these being typically ˜75% loss due to light saturation and photo-inhibition (PI) in the algae and ˜15% loss to respiration (growth, maintenance), making a feasible light to biomass conversion efficiency of only 1.9%.

It will be evident from the foregoing efficiency estimates that one of the key considerations are the dynamics associated with saturation and photo-inhibition which are linked to two fundamental and universal problems encountered when attempting to effectively culture algae for biomass: i) light intensity which is highly variable over time and ii) the absorption of light which is governed by Beer's law (for which see the discussion below).

There are huge variations in light intensity at the surface of the Earth and these occur over a vast range of timescales. Seasonal and diurnal changes and meteorological fluctuations mean that light intensity can vary over six orders of magnitude on a timescale ranging from milliseconds to hours—again six orders of magnitude. Alga can use regulation, acclimation and adaptation to respond to such changes on differing timescales: seconds to minutes (regulation), hours to days (acclimation) and years to aeons (adaptation) in a nested series of responses.

The photosynthesis process converts the electromagnetic energy of light to the electrical-potential energy of charge separation across a membrane to chemical energy in the form of fixed carbon. However, if excitation energy cannot be used directly it can result in the production of reactive oxygen species (e.g. via Chlorophyll triplet states) and cell damage or even death. There is therefore a fine balance between maximised efficiency, production rates and damage.

With regulation, algae are able to adjust components of the photosynthesis process rapidly and reversibly e.g. by regulation of enzyme activity (Rubisco) and by instigating light harvesting complex state changes from PSII to PSI. Thus the rates of photon capture, electron flow through the electron transport chain and the Calvin Benson Bassham cycle can be balanced (“poised”), at little material cost and on short timescales which minimise “downtime” losses (losses due to sub-optimal photosynthesis rates during the adjustment or recovery phase). However, regulation can only establish equilibrium between photon capture, electron transport and metabolic processes across a relatively narrow range of light intensities—the dynamic range. If the incident light intensity increases above this, the excitation pressure also rises and must be vented. This can take place constructively, whereby some of the excitation energy is utilised in electron sinks and is here termed non-carbon photochemical quenching (NCPQ). If these methods cannot cope, dissipatory methods must be used to prevent excitation build-up and damage and are termed non-photochemical quenching. Here the excitation pressure is converted harmlessly to heat or fluorescence in the Xanthophyll cycle (VAZ Dd/Dt) or by down-regulating the reaction centres. Finally photoinhibition is used to disassemble the reaction centre to prevent damage. Ultimately photo damage can occur if protective methods are incapable of venting the excitation pressure. Dissipatory methods and photo damage are both associated with a recovery period and significant downtime losses.

Acclimation is the process whereby the niche width for an alga is increased by realising longer term changes to elements of the photosynthetic apparatus. These include changes in antenna size (Chlorophyll molecules per light harvesting complex), the reaction centre density in membranes, PSI:PSII stoichiometry and changes in pool sizes. Such changes are associated with an energy and/or material cost and the alga is subject to sub-optimal photosynthesis rates until the changes are implemented.

Adaptation is the process by which inherited genetic traits result in optimised properties for a particular alga in a particular niche and can take generations to aeons.

Algal regulation processes explain the form of the PI curve typically exhibited by algae.—The PI curve FIG. 1 shows the photosynthesis rate (measured e.g. by O₂ production, biomass production etc.) as a function of light intensity. Initially, the photosynthesis rate increases linearly with light intensity (light dependent region—the dynamic range), followed by a tailing off of production towards the saturation intensity Iₖ where the graph becomes non-linear. At even higher intensities the photosynthesis rate reaches a maximum Pₘₐₓ when the graph goes horizontal due to saturation dynamics above Iₖ. At very high intensities photoinhibition and photo damage can occur and output is reduced below Pₘₐₓ. Adaptation, acclimation and regulation thus serve to balance light absorption, electron transport and the Calvin Benson Bassham reactions to optimise (but not necessarily maximise) photosynthesis across a huge range of irradiances.

Direct sunlight has around ten times the intensity of overcast light so a commercially viable PBR must be able to exploit direct sunlight effectively in order to maximise yields. In order to exploit high intensities, a high-light adapted strain should be used. This should be acclimated to the high light intensity conditions of the PBR—which is performed simply by circulating in the proposed PBR

The second fundamental issue is the absorption of light in media, which is dictated by Beer's law. In any light-absorbing medium the light intensity distribution obeys Beer's law if scattering is ignored. For an initial intensity Iₒ, absorption co-efficient α and a path length/the intensity distribution is:

I=I₀e^{-α·l}

or with a concentration N of absorbers with an absorption cross-section a the profile is

I=I₀e^{-·σ.l}

Here path length l refers to the distance travelled by the light, or the distance inside the growth medium aligned perpendicular to the sun (the z plane).

The light intensity reduces exponentially with distance travelled into the media, the light level is a maximum at the surface (I₀=100%), and rapidly reduces inside the media, reducing to −37% at one absorption length (α.l=1), 14% at 2 absorption lengths (α.l=2), and 5% at 3 absorption lengths (α.l=3). This is referred to as “canopy shading” or “self shading” as algae at the front shade those deeper inside the growth medium.

The average light intensity in a PBR with an optically dense growth medium (dark green) is significantly lower than an optically thin “light green” one. There are two extremes—a thick PBR with high absorption efficiency and low average light intensity or a thin PBR with high average intensity but low absorption efficiency.

One aim of the present invention is to provide a PBR which has a short optical path length, so as to provide a thinner, lighter PBR per unit area with less growth medium to pump.

There is no “optimum” PBR thickness in absolute terms (mm), it is instead dependent on the product of α.l or σ.N.l. Thus the same light intensity profile could be achieved in a thin PBR with a high concentration N as in a thick PBR with a low concentration N. Thus adjustment of the alga concentration N can be used to control accurately the intensity profile inside the PBR and vary the photo-modulation.

There are also spectral effects as a result of the wavelength dependent absorption of the various pigments in alga. Blue then red light is preferentially absorbed by chlorophyll a, with accessory pigments making additional contributions. The steepest intensity profile will be for blue light with green light exhibiting the flattest profile and the poorest spectral absorption efficiency.

In addition to dynamic variations in light intensity (diurnal/meteorological) there is ˜10³ variation in light intensity with algal growth medium depth, passing from the photic to aphotic zone in an optically dense growth media. Adaptation in nature means that to cope with the huge variety in intensity, alga find a niche at certain depths in the water column and use acclimation to widen that niche. In natural systems there is thus stratification, with high light (HL) adapted algae at the near the top of the water column and low light (LL) adapted algae deeper in the water column.

In the mass culturing of alga several approaches for PBR design have been used to accommodate the light intensity variation due to Beers law: the use of a two layer PBR with HL acclimated alga in the high intensity top layer and LL acclimated algae in the lower layer. The low-intensity light in the lower layer is predominantly green hence the use of e.g. rhodophyta which can effectively absorb and utilise green light. The downside is significantly greater complexity for a relatively small improvement in absorption.

An alternative approach to overcome the saturation effects at high intensities is to use genetic modulation techniques to truncate the light harvesting antenna of the reaction centres—by reducing the number of chlorophyll molecules from a few hundred to a few dozen.

Other systems actively exploit aspects of Beer's law to produce canopy shading in optically dense media which leaves cells in the rear in darkness. LL acclimated alga have very high photosynthesis quantum yields though are very sensitive to saturation/photo-inhibition. This is overcome by exploiting light dilution (light distribution) whereby portions of the growth media are constantly and successively cycled through the photic zone where they are exposed to high intensities for a short time and back into the aphotic zone for a recovery period in the dark. Thus for example ten times as many cells can be exposed per unit time, ensuring they experience 1/10^{th} of the overall intensity—thereby preventing saturation or photo-inhibition. This technique, however, necessitates moving large volumes of growth medium at high rates—requiring large energy inputs. Cells are also exposed to possible shear damage—plus the reservoir volume and therefore mass of the system is increased by an order of magnitude or more, necessitating high strength housing and support materials. With appropriate light intensities, flow velocities and PBR dimensions, cycle time and frequency can be optimised so that the alga in a PBR operates at high intensity without suffering photo-inhibition whilst exploiting the flashing light effect first discovered by Kok (“Experiments in photosynthesis by Chlorella in flashing light.”

The flashing light effect (FLE) has been used by researchers to increase optical conversion efficiencies in the lab using artificial light sources with a flash frequency and duty cycle which can be freely configured. It has been possible to exploit the FLE outside the lab in closed solar photo-bioreactors which exhibit canopy shading in optically dense growth media.

Two of the leading PBR designs exploit the FLE in solar systems (Green Fuel Technologies Corporation/Subitec GmbH) whereby sparged gas bubbles are used to drive mixing eddies in the growth medium. These eddies result in localised circular flow, and alga are moved to the front (light or photic portion) of the PBR then to the rear (dark) portion—transforming the spatial photomodulation to temporal photomodulation—so that the algae effectively experience intermittent light and dark conditions. Parameters of the PBR design (dimensions/use of baffles) and operation (gas flow rate) can be used to optimise frequency within a fairly narrow band. Such systems exhibit some of the highest photosynthesis rates and photosynthesis conversion efficiencies.

In open water, cloud cover results in a uniform spatial intensity profile below the surface in the X-Y plane, with an exponentially decreasing profile with depth (Z plane) which is dependent on algae concentration. The situation in direct sunlight, however, is very different due to the action of surfaces wavelets which are created by wind action which is almost permanently present. These wavelets refract light and with an approximately sinusoidal form they act as small positive lenses. In direct sunlight such lenses form a real inverted image of the sun in the water below them (caustics). Essentially the lens produces a beam of light converging towards the focus and then diverging away from it. As wavelets are essentially one dimensional, they are equivalent to one dimensional (1-D or cylinder) lenses which form a line focus or line images of the sun. The semi-random motion as waves travel and interfere with each other on the surface means that the line images are constantly changing and being scanned through the water below the surface.

The spatial pattern appears in nature (see FIG. 2) as a mesh of bright lines visible on e.g. the shallow sea floor, and in real time these are dancing profusely. The temporal intensity distribution experienced by alga at a fixed location corresponds to a burst or flash of high intensity light followed by a low intensity background. Such intensity profiles have been measured and analysed to identify the most probable durations as 10-30 ms. This effect is typically limited to surface waters whose depth is equivalent to the focal length of the wave lenses. Deeper water is below the focal point, the foci are blurred and enlarged such that the intensity of the radiation fluctuation decreases and the flash duration increases. Typical conditions for caustics formation are clear waters, high sun elevation and light winds.

For optimised alga culturing a PBR should optimise conditions for an optimised strain—that is to say one which is adapted to the local conditions (temperature, light levels) and whose cells are allowed to acclimate to these. The parameters should then be held as close to optimum as possible for as long as possible, i.e. temperature changes (ΔT) and intensity changes (ΔI) are minimum, such that regulation can rapidly accommodate the changes without energy/material costs or significant downtime required for acclimation. One way to reduce ΔI is to avoid the large changes in intensity due to absorption according to Beers law.

### SUMMARY

The present inventor seeks to provide improved photo-bioreactors and methods for cultivating biomass by photosynthesis which alleviate some or all of the problems and/or requirements set out in the foregoing and which may provide improved reactor efficiency.

According to various aspects of the present invention there are provided photobioreactors, and methods, for the cultivation of biomass by photosynthesis as set forth in the claims hereinafter and/or as set out in the following description. There is provided a bioreactor having a generally planar reservoir for containing a fluid growth medium while allowing incident light to penetrate the growth medium. In one aspect a plurality of lensing means are distributed in a fixed array above and generally parallel to the reservoir, each lensing means being adapted to concentrate light passing therethrough into the growth medium so as to form a correspondingly distributed plurality of regions of relatively high light intensity and relatively low light intensity in the growth medium. In another aspect a reactor base wall may be provided with a reflective surface to reflect unabsorbed light back through the growth medium. The reflection may be diffuse, specular or involve a Stokes shift. In another aspect a wall portion of the reservoir may include a gas membrane which facilitates gas transfer, in particular carbon dioxide into the reservoir and oxygen from the reservoir.

According to a general aspect of the invention there is provided a photo-bioreactor for cultivating biomass by photosynthesis which is provided with a reservoir for biomass through which a growth medium may flow and which is adapted by one or more of the features or aspects of the invention described or claimed hereinafter.

According to one aspect of the invention there is provided a generally planar photo-bioreactor for cultivating biomass by photosynthesis comprising:
a generally planar reservoir for containing a fluid growth medium while allowing incident light to penetrate the growth medium, and
a plurality of fixed lensing means distributed in a generally planar array above and generally parallel to the reservoir, each lensing means being adapted to concentrate light passing therethrough into the growth medium so as to form a correspondingly distributed plurality of regions of relatively high light intensity and relatively low light intensity in the growth medium.

Each lensing means may be adapted to form an associated focus. Preferably each lensing means is adapted to form an associated discrete focus in the growth medium. However it may be that one or more focus is formed at a location adjacent the growth medium but which is sufficiently close to concentrate light within the growth medium so as to form relatively light and relatively dark regions in the growth medium.

Each lensing means may comprise a refractive lens, a diffractive lens, an holographic lens, or a combination thereof. In a preferred arrangement each lensing means is adapted to form an elongate focus, preferably within the growth medium. When growth medium is induced to flow through the elongate focus in a direction oblique or perpendicular to the length of the foci the spatial photomodulation is converted into temporal photo-modulation within the growth medium, which is manifested as the flashing light effect thought to promote algal growth.

In a preferred arrangement the plurality of lensing means comprises an array of convergent lenses through which incident light passes before passing into the growth medium. The lens array is adapted to focus incident light into a plurality of foci located in the growth medium. The lensing means may comprise a plurality of elongate lenses (1-D lenses) which each provide a linear focus within the growth medium. The lenses can be straight or curved, or include multiple bends. However, preferably the elongate lenses are arranged in a generally parallel and/or side-by-side orientation, and preferably substantially co-planar with one another.

The focal length of each lensing means may be between 0.5 mm and 100 mm.

The lensing means array may be provided in or on a clear sheet material, and the clear sheet material may serve as a top wall (or upper wall) of the reactor.

In one embodiment each lensing means in the array may comprise a convergent lens element on one surface of the sheet and a divergent lens element on an opposite surface of the sheet, the divergent lens element being relatively less powerful than the convergent lens element.

Each lensing means in the array may comprise an aspheric surface and is preferably parabolic in form. This helps reduce image aberrations, so as to provide a sharply defined image in the growth medium.

The generally planar lensing means array is preferably spaced apart from the growth medium and located before the growth medium in the incident light path, such as by an intervening reactor compartment.

Fluid transport means should be provided for inducing a flow of growth medium along the reservoir so that the growth medium passes sequential regions of relatively high and relatively low intensity light so as to produce a flashing light effect in the growth medium. The fluid transport means are typically external to the reactor, for example pumps. They could however be internal, or could include gravity such as by location of the reactor on a slope. Recirculation of growth medium will however require transport back up hill.

In accordance with the foregoing aspect of the invention, by replicating surface wavelets and using growth media flow through the multiple foci, the spatial photo-modulation in the reservoir x-y plane ΔI(x,y) can be converted to temporal photo-modulation ΔI(t). Thus there is practically no spatial photo modulation, only temporal photo modulation, which is perceived as the flashing light effect—light energy is absorbed in the flash, and during the subsequent low-light period electron transport is performed and the Calvin Benson Bassham reactions fix CO₂ to produce G3P (the light-independent reaction—often erroneously referred to as the “dark reaction”).

In full sunlight there is temporal photo modulation the frequency of which can be controlled by adjusting the growth medium flow velocity, and whose duty cycle can be set by selection of lens spacing, focal length and proximity of the growth medium to the lens focus. This temporal photo modulation effectively “couples” the process of light absorption with the electron transport chain and the elements of the Calvin Benson Bassham cycle in the same way that a “work song” or sea shanty is used to synchronise movement in order to avoid clashing and enable elevated work rates and productivity. Temporal photo modulation therefore co-ordinates the timing of the various actions in the photosynthesis process, much the same as a cox in a rowing boat synchronises the rowers.

In a second aspect of the invention there is provided a generally planar photo-bioreactor for cultivating biomass by photosynthesis comprising:
(a) a reservoir for containing a fluid growth medium which allows incident light to penetrate into the growth medium and
(b) a light-reflecting surface disposed beneath the growth medium so as to reflect at least a portion of any light which has passed unabsorbed through the growth medium back through the growth medium.

This arrangement permits the bending of Beers law, as unabsorbed light is reflected back through the growth medium. Thus a higher average light intensity may be maintained in the growth medium (see FIG. 3, lines (ii) and (iii)). Deep portions of the growth medium receive a double dose of incident and reflected light of medium intensity, while the shallow portions receive a large dose of incident light and a small dose of reflected light.

Here pump light is absorbed as it passes through the crystal and a mirror is applied in order to reflect a portion of unabsorbed pump light back along its path such that more absorption can take place. This results in higher excitation densities, reduced photo modulation and higher absorption efficiencies.

The bioreactor as a whole, and preferably the reservoir and reflecting surface, are typically generally planar in form.

This aspect of the invention may be combined with any other aspect of the invention. Thus there is also provided a bioreactor as hereinbefore described in relation to the first aspect of the invention which also includes a light-reflecting surface which is disposed beneath the growth medium so as to reflect at least a portion of any incident light which has passed unabsorbed through the growth medium back through the growth medium.

Furthermore the light-reflective surface may be adapted for retro-reflective or Stokes shifted reflection of incident light which has passed through the growth medium. In the context of Stokes shifting, “reflection” includes the process of absorbing light at one wavelength and emitting light at another wavelength. The light reflecting surface preferably is provided by a base wall of the reservoir, which base wall preferably has a planar shape.

Thus in a preferred aspect of the invention a Stokes shifting medium is provided in the region of the light-reflective surface so that reflected portions of spectra not absorbable by algae on the first pass through the growth medium are shifted to frequencies which may be more readily absorbed by algae on the return pass back through the growth medium after reflection.

The Stokes shift may be effected by a high quantum yield fluorescent dye applied to, or included in, the light-reflective surface.

Furthermore, in systems with absorption characterised by a pronounced spectral dependency, light which is not absorbed on a pass can be Stokes shifted to wavelengths which are absorbed, and using the rear reflector this light can be directed back along the original light path.

A wavelength shifting agent is typically a fluorescent die which absorbs light at one wavelength and emits at a longer wavelength (Stokes shift=Δλ see FIG. 4) with a quantum yield defined by the number of photons emitted compared to those absorbed. By selecting an appropriate dye with high quantum yield which absorbs in the green portion of the spectrum (where Chlorophyll a containing algae typically have a low absorption coefficient) and emits in the red where chlorophyll has an absorption maximum, unabsorbed green light is made available for absorption to improve the spectral absorption efficiency.

By positioning the dye on the rear wall, before the reflecting surface, green light not absorbed during a pass through the alga in the growth medium is Stokes shifted to red light which is emitted directly into the growth medium or is emitted towards the rear of the PBR and then reflected by the rear mirror back into the PBR. Thus all stokes shifted light has to complete a full pass of the growth medium from back to front, with absorption again dictated by Beer's law. Now, however, the intensity profile of the Stokes shifted light is highest at the rear of the growth medium and reduces exponentially towards the front. The total intensity profile is composed of the incident light (travelling front to back) and the reflected/Stokes shifted light (travelling back to front) which combine to produce an intensity profile very different to the exponential profile of Beer's law. FIG. 3 shows the original Beers intensity profile (labelled Beers) in a notional reactor and the intensity profile obtained using the rear mirror to reflect light (Beers plus mirror). This, added to the intensity profile of the Stokes shifted light (Stokes shifted) combines to produce the uppermost profile with fairly uniform high intensities (Beers plus mirror and Stokes).

Thus Beer's law can be bent or even broken by using a rear reflector and appropriate selection of fluorescent dye and algae concentration—resulting in a total light intensity profile which is almost constant—i.e. spatial photo modulation in the Z plane is nearly zero, all algae experience the same light intensity regardless of depth, though with very high absorption efficiency and high average intensities. Thus the present invention provides a way of greatly reducing problems introduced by canopy shading.

The wavelength shifting Stokes compound can be incorporated into a clear thermoplastic sheet and bonded between the back wall and the reflective surface, or applied to the reflective surface in a bonding matrix. Alternatively the back surface could be formed from a thermoplastic sheet impregnated with wavelength shifting compound.

These techniques can result in more energy being deposited in the growth medium thereby increasing absorption efficiency. More importantly, this energy is deposited in the rear portion of the PBR which in prior art designs are dark. This results in a near-homogeneous intensity profile through the growth media, with reduced spatial photo-modulation in the z plane ΔI(z)˜0.

In any of the bioreactors in accordance with the various aspects of the invention a plurality of static mixing features may be distributed in the reservoir for inducing mixing of the growth medium when flowing in the reservoir. The mixing features may comprise a series of ridges, striations, recesses, fins, baffles or partial barriers formed on or in a reservoir wall surface.

In a third aspect of the invention there is provided photo-bioreactor for cultivating biomass by photosynthesis comprising:

(a) a transparent top wall for admitting incident light into the reactor,

(b) a base wall disposed below and spaced apart from the upper wall, and

(c) a transparent dividing wall disposed between the top and base walls and spaced apart therefrom, which dividing wall defines an upper wall of a reservoir for fluid growth medium defined between the base wall and the dividing wall, and a lower wall of an upper compartment defined between the dividing wall and the top wall.

Preferably the photo-bioreactor has a generally planar configuration. For example the top wall, base wall and dividing wall may each form generally planar layers in a reactor.

The third aspect of the invention may be combined with any of the other aspects of the invention. Hence there is provided for example a bioreactor as hereinbefore described with reference to the first and/or second aspects of the invention comprising:

(a) a generally planar transparent top wall for admitting incident light into the reactor,

(b) a generally planar base wall disposed below and spaced apart from the upper wall, and

(c) a generally planar transparent dividing wall disposed between the top and base walls and spaced apart therefrom, which dividing wall defines an upper wall of the generally planar reservoir for fluid growth medium defined between the base wall and the dividing wall, and a lower wall of an upper compartment defined between the dividing wall and the top wall.

Thus the generally planar transparent top wall for admitting incident light into the reactor may have therein or thereon a distributed array of lensing means. The generally planar base wall may provide a generally planar light-reflecting surface disposed beneath the growth medium.

The top wall is preferably structurally rigid, which is to say it should be self-supporting or capable of supporting its weight without unduly sagging. Similarly the base wall is preferably structurally rigid. Side walls are incorporated at regular intervals to enable the top, dividing and base walls to be constructed from light-weigh materials, with the box-sectioned profile giving rigidity to the whole.

The upper compartment is occupied by a fluid, such as air, an inert gas or a liquid such as water. The lensing means (when present) may be adapted to provide foci which fall above the growth medium or reservoir when the upper compartment contains air and below the growth medium when the upper compartment contains water.

In a fifth aspect of the invention the bioreactor upper compartment communicates with fluid management apparatus. This apparatus permits introduction and removal of gas or liquid in the upper compartment so as to promote heat exchange from the growth medium to the upper compartment and thence to the environment. Alternatively, or in addition, the fluid management apparatus permits the partial or complete emptying of one heat exchange fluid in the upper compartment and replacement with another heat exchange fluid so as to permit tailoring of heat exchange between the growth medium and heat exchange fluid in the upper compartment by selection of a replacement fluid which has a desired thermal conductivity and specific heat capacity, and wherein the apparatus preferably comprises a fluid pump and one or more sources of heat exchange fluids.

In one arrangement the upper compartment is subdivided into discrete channels which may be filled with the same or different fluids. For example, the reactor reservoir region may be provided with internal wall portions which subdivide the growth medium reservoir into a plurality of channels. The internal wall portions (and optionally a suitable manifold) may define a tortuous path for growth medium when conveyed through the reservoir. Preferably the internal walls define generally parallel channels for conveying growth medium. The channels may be fed by one or more distribution manifolds and/or feed one or more collection manifolds, with flow in adjacent channels in the same direction (parallel) or opposite (tortuous/serpentine).

In a sixth aspect of the invention there is provided a photo-bioreactor for cultivating biomass by photosynthesis wherein at least a portion of a base reactor wall defining the growth medium reservoir comprises a gas-permeable membrane and a gas conduit is defined juxtaposed to the gas-permeable membrane so as to permit transfer of oxygen from the growth medium into the gas conduit and/or carbon dioxide from the gas conduit into the growth medium. This aspect of the invention may be combined with any other aspects of the invention described herein.

Thus the reactor may have a reservoir enclosure for containing a fluid growth medium, and a gas conduit juxtaposed to a reservoir base wall portion, wherein at least a portion of the reservoir wall portion bordering the growth medium comprises a gas-permeable membrane adapted to permit transfer of oxygen from the growth medium into the gas conduit and/or carbon dioxide from the gas conduit into the growth medium.

The gas conduit conveniently comprises a reactor compartment formed below a base wall of the reservoir. Said reactor compartment is preferably generally planar in form. Means are preferably provided for transporting a gas mixture past the gas-permeable membrane so as to facilitate gas exchange with the reservoir.

In a preferred arrangement means are provided for transporting growth medium in the reservoir, and the means for transporting the gas mixture in the gas conduit or compartment is adapted to convey gas in a direction substantially counter-current with respect to the direction of flow of the growth medium.

The gas-permeable membrane is typically a microporous membrane, which permits the passage of gas or vapour, but not liquid. The microporous membrane may form part or all of a reservoir base wall and may serve to provide a light-reflecting surface so as to reflect any portion of light which has passed unabsorbed through the growth medium back through the growth medium, in accordance with the second aspect of the invention described hereinbefore.

The microporous membrane serves as a diffuse reflector and is preferably selected to provide a total reflectivity of greater than 95%, preferably greater than 98%. The microporous membrane is typically an hydrophobic membrane, optionally an oleophobic membrane.

A ratio of gas-permeable membrane surface area to growth medium (or reservoir) volume per unit length of the reactor may be greater than 100 m²/m³, preferably greater than 400 m²/m³.

In a seventh aspect of the invention there is provided a photo-bioreactor for cultivating biomass by photosynthesis, preferably by solar light, which comprises an elongate transparent generally planar member which serves as a dividing wall between a generally planar reservoir for growth medium on one side and a further generally planar compartment for a heat exchange fluid on the other side of the dividing wall, wherein the reactor structure is sufficiently flexible to permit rolling of the reactor onto a spool, for storage or transport and deployment of the reactor by unrolling from the spool.

With gas transfer occurring along its length, the reactor length is not limiting such that it is linearly scalable and may be formed in any desired length. Spooling facilitates installation by unreeling onto a prepared or flat substrate, such as ground. The substrate itself may be inclined towards the sun, by for example use of a man-made structure or frame, or by taking advantage of natural topography.

There is also provided a photo-bioreactor as hereinbefore described which comprises an elongate transparent generally planar member which serves as a dividing wall between a generally planar reservoir for growth medium on one side and a further generally planar compartment for a heat exchange fluid on the other side of the dividing wall, wherein the reactor structure is sufficiently flexible to permit rolling of the reactor onto a spool, for storage or transport and deployment of the reactor by unrolling from the spool.

The reservoir may comprise a generally planar base wall sheet disposed under the dividing wall and a generally planar transparent top wall which is disposed above the dividing wall, and wherein sidewalls are provided which extend generally orthogonally between the respective walls. The sidewalls may comprise a flexible material, preferably closed-cell foam material, such as neoprene or rigid plastics

The base wall may comprise a gas permeable membrane and a further generally planar compartment may be disposed under the base wall to define a gas exchange conduit or compartment beneath the base wall. The top wall may comprise a plurality of elongate convergent lenses disposed side-by-side and extending generally transversely or obliquely across the front sheet.

In any of the bioreactors hereinbefore described the reactor is preferably elongate. A general direction of growth medium flow may be along the length of the reactor, whether in parallel, or following a tortuous/serpentine or switch-backing path. For example the reactor may be elongate and wherein the growth medium reservoir may be divided into a plurality of channels.

In any of the reactors hereinbefore described one or more of: structural walls, dividing walls and planar walls thereof and which make up the reactor may comprise a transparent material having a refractive index of less than 1.40, preferably less than 1.36. In particular at least a portion of the reactor structure which in use abuts aqueous growth medium or aqueous heat exchange fluid comprises a material having a refractive index of less than 1.40, preferably less than 1.36. In one embodiment the transparent material is a fluoropolymer, for example a fluorinated ethylene propylene or a poly(tetrafluoroethylene).

The reactors of the invention are preferably closed solar reactors, but the technology may be applied to certain open reactors, or closed reactors having an artificial light source.

In another aspect of the invention there is provided a method of cultivating biomass by photosynthesis comprising:
providing a photo-bioreactor as hereinbefore described,
introducing into the reservoir a fluid growth medium in which photosynthetic algae are dispersed,
exposing the bioreactor to incident light so that at least a portion of the incident light penetrates the growth medium so as to promote growth and reproduction of the algae.

The growth medium will in practice be caused or allowed to travel through the reservoir, and is preferably re-circulated. The growth medium may travel with a velocity of less than 0.3 m/s, preferably less than 0.1 m/s. The growth medium may be caused to flow in a manner in which turbulence is formed so as to enhance local mixing of the growth medium to improve mass transfer.

A planar fluid compartment disposed above the reservoir may be charged with a heat exchange fluid. The fluid may serve to insulate the growth medium in the reservoir from ambient temperatures, in particular extremely hot or cold conditions. Alternatively the fluid may serve to cool the growth medium by heat conduction. According to conditions, the fluid may be replaced by a substitute fluid having a different thermal conductivity, specific heat capacity or temperature.

The fluid compartment may have a cross-section which is subdivided to form a plurality of channels. One or more of the channels may be charged with a first fluid and one or more of the other channels is charged with a second fluid having a different thermal conductivity, specific heat capacity and/or temperature, thereby to provide a graded insulation or heat transfer performance. For example one fluid may be gaseous and the other liquid.

The biomass cultivation is conducted in an hermetically closed system so that the algae and growth medium are isolated from the ambient atmosphere. Thus the bioreactor may be a closed bioreactor. The incident light is preferably provided by solar radiation.

The bioreactor may include a gas-exchange conduit which is separated from the growth medium reservoir by a gas-permeable membrane. A gas mixture comprising carbon dioxide may be present in the said conduit. Oxygen in the growth medium may be allowed to transfer from the growth medium through the membrane to the gas exchange conduit. Carbon dioxide may be allowed to transfer from the gas exchange conduit through the membrane and into the growth medium to replace carbon dioxide depleted by photosynthesis. The gas mixture may be air, optionally with raised levels of carbon dioxide. The gas in the exchange conduit may be induced to flow in a direction which is counter-current to a growth medium flow direction.

In a preferred embodiment of the present invention there is provided a method of cultivating biomass by photosynthesis comprising providing a fluid growth medium in which biomass is dispersed and which light can penetrate, exposing the growth medium to a source of incident light characterised in that the incident light is lensed using lensing means so as to form one or more elongate light foci in the growth medium, and transporting growth medium through said foci, preferably in a direction transverse or oblique to a longitudinal foci axis, so as to provide temporal photo-modulation. The process may be carried out using any suitable bioreactor, but especially those hereinbefore described.

The lensing means may selected from one or more of: refractive lenses, diffractive lenses, holographic lenses, or combinations thereof. Simple refractive lenses are preferred as these provide a low cost manufacturing solution. The lensing means is preferably adapted to form one or more elongate foci in the growth medium. Thus the lenses mimic the effect of wavelets generated by wind on exposed water.

The lensing means may in a simple arrangement comprise an array of convergent lenses through which incident light passes before passing through the growth medium. The lens array should be adapted to focus incident light into a plurality of foci located in or near the growth medium.

The lens array may comprise a plurality of elongate lenses which each provide a linear focus within or near the growth medium. For example each lens may have the form of a chordal spherical section of a cylinder. Other sections are possible, such as aspherical, sinusoidal or parabolic. The elongate lenses may be arranged in a generally parallel and/or side-by-side orientation similar to a lenticular array.

A converging lens forms a real, inverted image of a distant object at its focal plane. The distance from lens to the focal plane is termed the focal length and is approximately equivalent to the image distance. In the current arrangement, this distance is provided by an optically inert top PBR layer—a layer of gas or water through which the light passes as it converges to a focus. Thus in the specific embodiment a two layer PBR structure is used. The upper layer conveniently provides a compartment or chamber for thermal management.

In heat dominated climates the inert layer is filled with water to increase the thermal conductivity and heat loss to the environment. This water also acts as a thermal mass to reduce growth medium temperature increase. In cold dominated climates, the inert layer is filled with air or other fluids to reduce thermal conductivity and heat loss to the environment in order to hold the growth medium temperature high. Intermediate temperatures can be accommodated by partially filling the layer with liquids for fine control.

In a preferred arrangement the lens array is provided on a clear sheet material. The clear sheet may be formed with the lens array by extrusion, embossing or roll-casting of a clear polymer plastics material.

In one embodiment, each lens in the array comprises a convergent lens element on the top surface of the sheet and a divergent lens element on the bottom opposite surface of the sheet, the divergent lens element being relatively less powerful than the convergent lens element. Choice of suitable radii of curvature and image distance ensure that the focal length, i.e. the position of the focus in the growth medium, is preserved when the refractive index of the inert PBR upper chamber is changed from air (RI=1) to water (RI=1.33). Thus the thermal properties can be altered without significantly altering the optical properties of the PBR.

The lens array may be spaced apart from the upper wall of the growth medium and located before the growth medium in the incident light path.

For a simple recumbent (lying parallel to the ground) system in equatorial regions the lens arrays are made up of a plurality of elongate (1-dimensional) lenses which are preferably aligned East-West with growth medium flow in the reactor North-South. Here solar elevation is always about 90 degrees and the sun tracks from E to W passing overhead at noon. Thus the 1-D lenses in the reactor are relatively unaffected by variations in solar azimuthal angle.

For a recumbent system in higher latitude regions, azimuthal changes may be accommodated by the 1D lenses, though solar elevation will always be off the ideal incident axis of the lens leading to potential lens aberrations, enlarged foci and reduced temporal photo-modulation (PM_{T}). To overcome this, in a particular embodiment, each lens in the array may be angularly offset towards the average solar elevation, for example by adopting a “factory roof” configuration in a front sheet on which the lenses are disposed. Again, preferably with lenses aligned E-W and growth medium flow N-S. In this way rays in the morning and evening are slightly below the axis, and at midday are slightly above the axis, resulting in minimised total aberrations from off-axis rays throughout the day.

At high latitudes, tilting reactor systems can be used in which the reactor may be tilted over time to ensure that solar rays are always on or near the axis. The tilting system can be arranged to continuously track the solar elevation, track stepwise, or have a fixed angle. Two geometries are possible with tilting systems:

With growth medium flow in the horizontal plane the lenses are aligned N-S down the inclined PBR and the growth medium flows E-W through the line foci. This arrangement permits long PBR lengths, though with suboptimal lensing performance early a.m. and late p.m. as variations in azimuthal angle cannot be accommodated. However, light intensities tend to be low at these times and FLE is less important.

(ii) With growth medium flow in the vertical plane, the lenses are aligned E-W and GM flow is aligned N-S and passes up/down through the inclined PBR. This arrangement can accommodate changes in azimuthal and elevation angle, though practical considerations limit the PBR height.

In another aspect of the invention there is provided a method of cultivating biomass by photosynthesis comprising providing a fluid growth medium in which biomass is dispersed and which light can penetrate and exposing the growth medium to a source of incident light, characterised in that a portion of the incident light passes through the growth medium and is reflected back through the growth medium by means of a light-reflecting rear surface.

By using a reflective rear surface the absorption efficiency of a reactor may be increased and spatial photomodulation decreased. This is because the reactor thickness and growth medium density may be adjusted so that sufficient unabsorbed light remains after one pass to allow the reflected light to pass back through the growth medium. Thus at the back of the reactor the light intensity is effectively doubled resulting in a more uniform light intensity through the reactor. The flashing light effect may be used by providing a strobing light source in accordance with the prior art, or by the use of the lensing approach of the present invention, in which the flowing growth medium is exposed to periodic relatively high intensity light bursts as foci are encountered followed by low light intensities in the dark regions between the foci.

Thus Beer's law can be manipulated using a reflective reactor base wall to allow relatively low spatial photo-modulation (PM_{S})though retaining good absorption efficiencies and the orthogonal flow of the growth medium through the foci produced by optical elements establishes temporal photomodulation (PM_{T}) with the frequency determined by lens element spacing and growth medium flow velocity, the latter can be adjusted according to light intensity—high velocity/frequency for high light intensities and low for low light intensities.

The light-reflecting surface may be adapted to provide specular reflection, for example by mirroring. Alternatively, the light-reflecting surface may be adapted to provide diffuse reflection (e.g. multiple reflections from air: dielectric interfaces in a microporous or foam material, via application of light reflecting compounds such as white paint, or through the use of retro-reflective coatings (e.g. microspheres which reflect light back along the incident path, preserving any spatial profile due to lensing). In each case at least 90% of the incident light is reflected back into the growth medium. Typically the reflectivity of the surface is at least 95%. The surface will typically lie at a solid/liquid (growth medium) interface, which enhances the reflectivity as compared to an air/solid interface (˜4%) or liquid/solid interface (˜1%).

Growth medium translation means may be provided for continuously moving growth medium through the light foci. The movement should preferably be such that the direction of travel is perpendicular to the orientation of the elongate foci so that cells in the liquid pass through the foci.

The growth medium moves with a velocity of less than 0.3 m/s, preferably less than 0.2 m/s. A target velocity is about 0.1 m/s in bright sunlight. This is somewhat less than is usual in the art. Without direct sunlight (10× reduction in intensity) the velocity can be reduced by an order of magnitude or more.

For a threefold reduction in growth medium flow, the power requirement (which has a cubic dependence on flow velocity), will be 27 times smaller. Factoring in a 10× reduction in growth medium volume (photic zone only) and two fold reduction in depth (two absorption paths in one physical length) similar absorption efficiencies can be achieved in system requiring ˜540 times less mixing energy and with 20 times less mass.

One or more liquid mixing or deflecting features are provided for inducing mixing in the moving growth medium. This ensures mixing of the growth medium to promote gas and nutrient exchange. The mixing features may comprise surface discontinuities provided in a growth medium container wall, such as a series of ridges or striations formed in the wall surface.

The cultivation should take place in an hermetically closed system in which growth medium is isolated from the ambient atmosphere. This prevents extraneous agents, such as competing alga, grazing zoo plankton or pathogens disrupting biomass growth.

In the present innovation incident light is preferably provided by solar radiation. However in theory artificially generated light could be used, and this may be commercially feasible if high value biomass is being grown under tightly controlled conditions.

The fluid growth medium is preferably a liquid, and in practice will be aqueous. The preferred biomass is a phytoplankton or micro algae.

In yet another aspect of the invention there is provided a photo-bioreactor for photosynthesis of biomass comprising a portion for containing a fluid growth medium while allowing incident light to pass through the growth medium and a light-reflecting surface which is disposed so as to reflect light which has passed through the growth medium back through the growth medium. This embodiment may stand alone or be combined with the lensing aspect hereinbefore described.

The light-reflecting surface may be adapted to provide specular reflection, for example by mirroring. The light-reflecting surface may be adapted to provide diffuse reflection, such as by the use of a multitude of clear thermoplastic/air interfaces (microporous membrane/foam) or the provision of a white coating or other colouration.

In yet a further aspect of the invention there is provided a photo-bioreactor for photosynthesis of biomass in a reactor as hereinbefore described which has a generally planar, layered layout with an upper inert chamber and a lower chamber containing the growth medium. For the growth medium chamber, an upper sheet is disposed above and spaced apart from a base sheet, with growth medium disposed between the upper and lower layers.

The upper sheet of the inert chamber may be provided with the lensing means. The base sheet of the growth medium chamber may have an upper surface which is adapted for specular or diffuse reflection of incident light. An intermediate sheet may be disposed between the upper and base layers, which intermediate sheet defines in the reactor a lower compartment in which is disposed growth medium and an upper compartment which separates the upper sheet from the growth medium and acts as the image distance for the lensing elements such that the foci fall in or near the growth medium below. The upper compartment may be occupied by a variety of heat transfer fluids for regulating thermal conductivity and heat loss, such as air, an inert gas (e.g. neon or argon) or a liquid (e.g. water). Thus in a warm environment with bright sunlight and high solar gain, water is used in the upper compartment to increase thermal conductivity and heat loss, whereas in cooler environments air or even Argon are used to reduce thermal conductivity and heat loss, to provide optimum growth medium temperature. Additional temperature regulation can be provided by passing the growth medium through a heat exchanger with appropriate external heat or cold sinks.

In typical reactors a focal length of the lenses or lensing means may be between 0.5 and 100 mm, preferably 1 mm to 10 mm. Smaller or longer focal lengths may be used according to requirements. In any case the focus should preferably fall in the growth medium above the base sheet, although the focus may fall outside the growth medium provided that regions of concentrated light are formed in the growth medium. Thus, as compared to the intensity of incident light falling on the reactor, areas of higher intensity and relatively low intensity will be formed in the growth medium.

The reactor may be provided with internal walls. These walls support the base and top sheets and can also define a tortuous path for the growth medium through the reactor. For example, the base sheet may be provided with upstanding internal wall portions which define a tortuous path for growth medium when conveyed through the reactor. Alternatively, or in addition, a manifold may be used to convey fluid into a plurality of parallel channels defined by the side walls.

Following is a description by way of example only and with reference to the figures of the drawings of ways of putting the invention into practice.

### BRIEF DESCRIPTION OF THE DRAWINGS

FIG. 1 is a graph showing the saturation and photo-inhibition of algae growth rate as light intensity increases.

FIG. 2 is a photograph taken undersea and showing the array of solar images formed by surface wavelets.

FIG. 3 shows the intensity profile with simple absorption for rays travelling front to back in bioreactors.

FIG. 4 shows the absorption and emission spectra of a fluorescent dye and the resulting Stokes shift—the difference in wavelength between the maxima.

FIG. 5A is a perspective view from above and one side of a photobioreactor according to the present invention.

FIG. 5B is a perspective view of a portion of the bioreactor sectioned along the line BB′ shown in FIG. 1A.

FIG. 5C is an enlarged view of the area designated C in FIG. 5B.

FIG. 6 is a schematic plan view of the photobioreactor of FIG. 5, showing an example of the flow path of growth medium in the reactor.

FIG. 7A is a schematic representation of an incident light path in the growth medium.

FIG. 7B is a schematic representation of a reflected light path.

FIG. 7C is a schematic representation of both incident and reflected light paths in the growth medium.

FIG. 7D is a schematic representation of incident, reflected and Stokes shifted light paths in the growth medium.

FIG. 8A is a schematic representation of light paths through a transverse cross-section of the reactor, showing the effect of lensing elements on parallel rays from the sun showing the multiple foci.

FIG. 8B is a graph showing the resulting light intensity in the reactor growth medium as a function distance X along the reactor chamber, showing the regular peaks associated with the foci, with the peak-peak distance equal to the lens spacing.

FIG. 8C is a graph showing the resulting temporal light intensity experienced by algae cells in the reactor growth medium as they travel in the X direction. The spatial photomodulation PM_{S} is converted to temporal photomodulation PM_{T} as a result of the flow velocity in the X direction. The frequency of the temporal photomodulation is therefore dependent on lens spacing and flow velocity, and for a given PBR construction can be adjusted by adjusting flow velocity.

FIGS. 9A, 9B, 9C and 9D show various PBR arrangements, lens orientations and growth media flow directions for tilting (A,B) and recumbent (C,D) arrays.

FIG. 10 is a schematic representation of an algae growth system in which a reactor according to the invention is incorporated.

FIG. 11 is a perspective front three quarter view of a sectioned bioreactor according to a second embodiment of the invention.

### DETAILED DESCRIPTION

In this detailed explanation, the subject matter mechanism is explained with references to figures in order to make the subject matter more understandable without forming any restrictive effect. Accordingly, in the explanation below and in the subject matter figures, the subject matter invention is assumed to be applied in providing movement of the ram of a press. However, in alternative embodiments, the subject matter invention can also be used in any field where the rotational movement is required to be transformed into different movements.

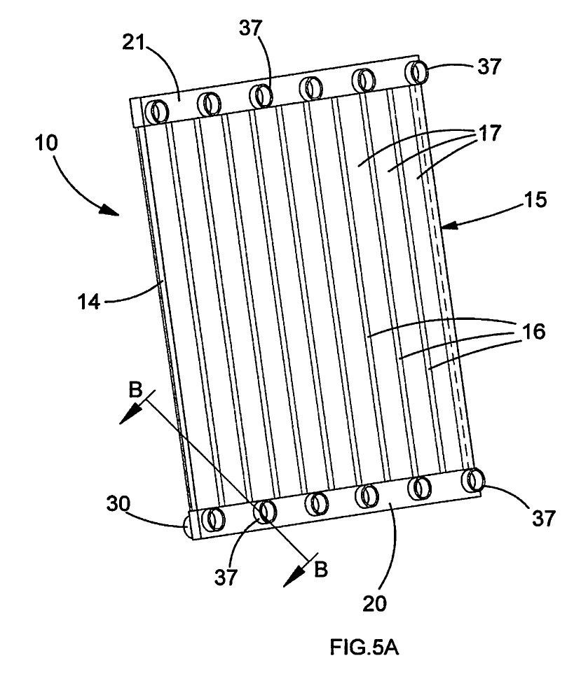

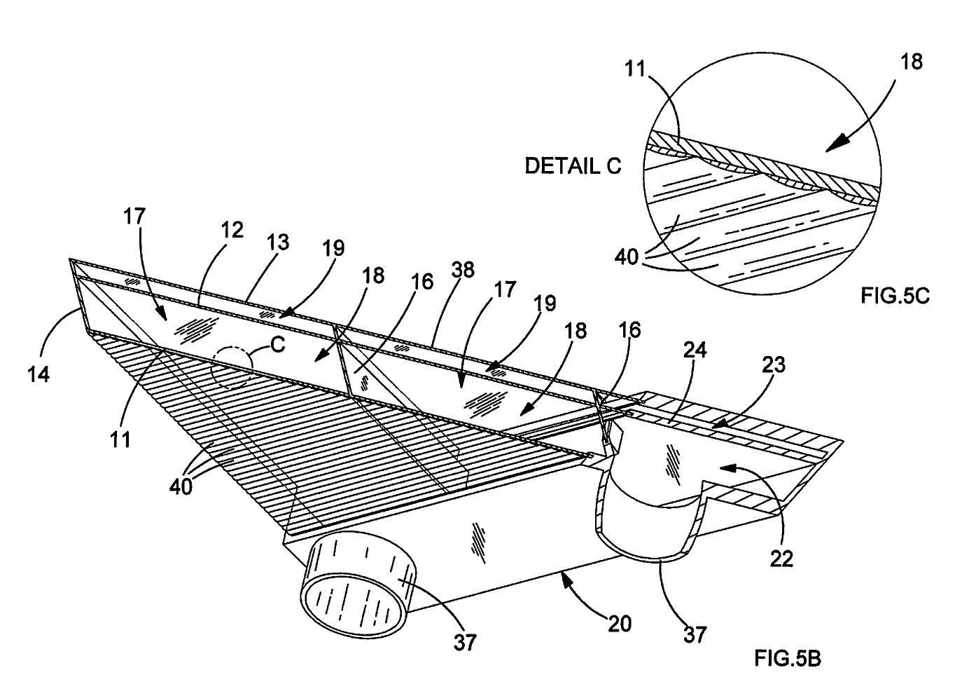

In FIG. 5A a first embodiment photo-bioreactor for photosynthesis of algae is shown generally as 10. The reactor is rectilinear and planar in configuration. The reactor has an upper planar wall 11, a middle planar wall 12 and a base planar wall 13. Upstanding orthogonal sidewalls 14, 15 are provided at each side of the reactor. These sidewalls extend from the base wall to the middle wall and up to the upper wall. A series of parallel internal dividing walls 16 are provided in the reactor. Each of the internal walls extends vertically from the base wall via the middle wall and to the upper wall, and along the long axis direction of the reactor. In the embodiment of FIG. 5A, nine internal side walls are shown. Together with the sidewalls, these internal walls define ten internal parallel elongate flow channels 17 in the reactor. Each channel is divided into upper 18 and lower 19 internal compartments by the middle wall, which acts as a reactor internal divider.

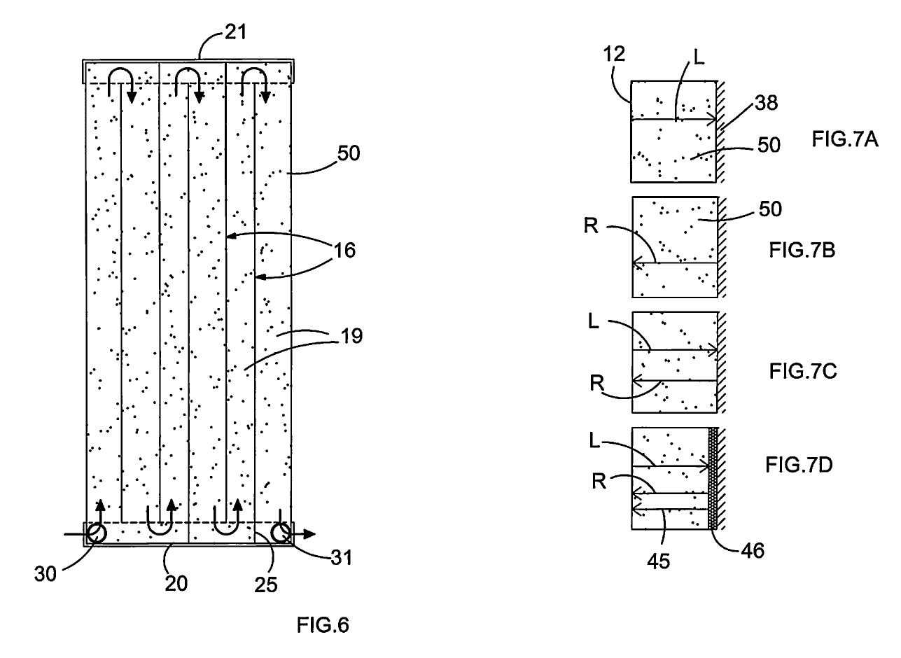

Proximal and distal manifold caps 20, 21 are attached to proximal and distal end regions of the reactor. The end caps each have an internal manifold volume, which is divided into upper 22 and lower 23 chambers by a manifold divider wall 24. The lower manifold chamber 23 is itself divided by internal walls (not visible in FIG. 5) which are disposed as continuations of internal divider walls 16. However, and as shown in FIG. 6 (which is a plan view of a section through the lower chamber 19 of the reactor and manifold chamber 23) the end portions of the internal walls 25 can optionally be alternately omitted and retained 27 so as to provide a tortuous path in the reactor upper and lower compartments for fluid flowing therein. Note that only six flow channels are shown in FIG. 6 for the sake of simplicity.

The upper manifold chambers 18 and 22 (shown in FIG. 5B) are fed and drained by one or more spaced apart upward facing ports 37 formed in the manifold cap 20, 21 upper walls. These ports allow the fluid (gas or liquid) in the upper chambers 18, 22 to be changed according to requirements for temperature regulation: in cold climates/at night, low thermal conductivity fluids can be used (air or argon) to insulate the reactor whereas in hot climates or during high solar gain due to intense sunlight, high thermal conductivity fluids are used (helium or water) to increase heat loss and limit the increase in growth medium temperature. Using mixing features (such as vortex generators, fins or striations—not shown) in the upper chambers 18,22 and circulating the high thermal conductivity fluid will cause eddies to form which will further enhance heat loss to the environment.

The base wall 13 is formed of thermoplastic material provided with a mirror coating. Alternatively a diffuse reflective coating or retro reflective or Stokes shifting coating. The middle dividing wall 12 is generally formed of clear thermoplastic material. The sidewalls 14, 15 and internal walls 16 are composed of thermoplastic material or foam.

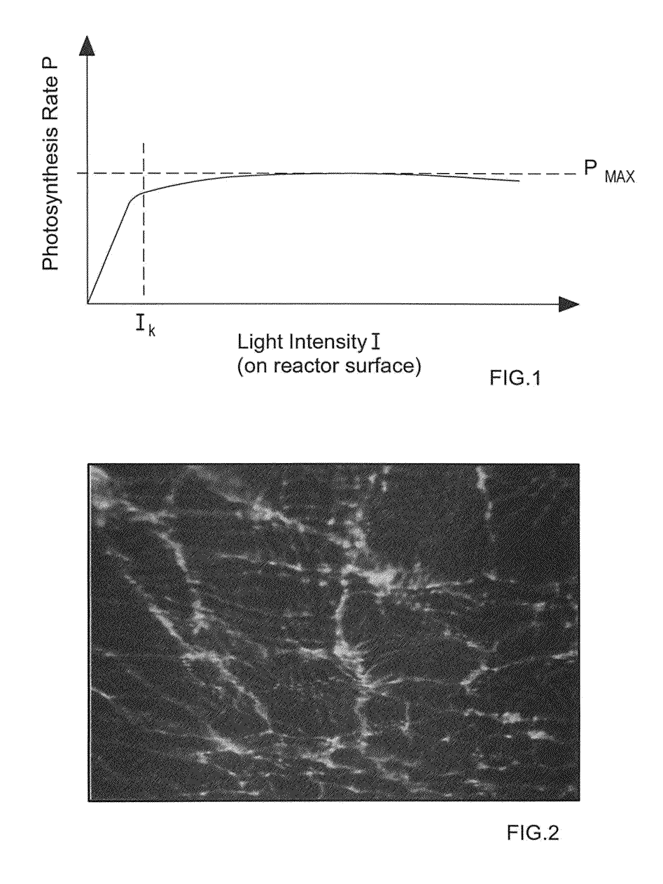

The upper wall 11 is formed of clear thermoplastics sheet material. An upper surface of the upper wall is formed with a plurality of parallel elongate transverse lenses 40, shown in FIG. 1C. The lenses each have a section which is essentially parabolic or spherical and represents a minor chordal portion of a circle. The lenses may be applied for example by embossing using a rolling mill. Alternatively an array of Fresnel or holographic lenses may be used.

The exact shape of the lens varies depending on depth of growth media chamber and upper chamber and refractive index of the upper wall. The lens spacing is dependent on the desired frequency for exploiting the flashing light effect, though is typically of the order of 0.1-1 mm to enable frequencies of 1000-100 Hz with a growth media flow velocity of 0.1 m/s. The radius of curvature is chosen such that the lens has a focal length approximately equal to or just above the upper chamber depth such that the focus is formed in the growth media, which is thin with a high cell concentration and high absorption coefficient such that most light is absorbed before the light can diverge away from the focal plane. For a 10 mm deep upper chamber filled with air, an upper sheet with a R1 of 1.5 whose lower surface is planar (infinite radius of curvature) the required radius of curvature is derived from

$$\frac{1}{f} \approx ( n - 1 ) [ \frac{1}{{R}_{1}} - \frac{1}{{R}_{2}} ] .$$

Which for R2=∞, n˜1.5, f=10 mm yields R1=5 mm.

The lenses extend transversely from sidewall 14 to opposite sidewall 15.

The manifold end caps 20, 21 are formed from e.g. moulded opaque structural plastics material and may include reinforcing additives such as glass fibre lengths. An inlet port 30 is provided on the manifold cap 20 of the reactor. An outlet port 31 is provided through the same manifold cap or on the other manifold cap. The ports 30,31 and feed/discharge the lower internal reactor chambers 19, 23. Alternatively a simple manifold can be applied to the base and upper layers. This has e.g. box or rectangular section with holes placed at regular spacing which match holes in the upper and base layers and allow fluid flow into the reactor chambers.

A growth medium for use in the lower reactor/manifold chambers 23, 19 is an aqueous formulation which comprises water, algae and nutrients. The growth medium has a typical composition as follows: algae cells, density range from 0.1-5% v/v, macro nutrients nitrate, phosphate, potassium, sulphate, silicate and micronutrients (trace elements plus vitamins B1 and B12 (e.g. Walne medium or Guillard's F/₂ medium)). An exemplary growth medium composition is given hereinafter.

In use, the growth medium 50 is fed into the reactor via the inlet port 30 of the manifold. The growth medium flows through the lower channel compartments and out of the distal manifold, or can be switched back along adjacent channels as shown in FIG. 6.

A mirror, diffusely reflective, or retro reflective layer (38) is provided on the top (inside) surface of the base wall 13 of the reactor. In some embodiments the reflective layer is a Stokes shifting layer. The Stokes shifting layer in this embodiment is a clear polymer matrix in which is dispersed a fluorescent dye. The dye absorbs light which passes through the growth medium and emits the absorbed light at a longer wavelength which can be absorbed by the algae in the return pass through the growth medium.

In FIG. 7D the effect of a Stokes shift coating is shown. The incoming light is reflected as for FIG. 7C, but a proportion of the light which is not capable of being absorbed by algae in the growth medium is absorbed by a Stokes shifting agent layer 46 in front of the mirror. This agent fluoresces to emit light of a different, shifted wavelength which can then be absorbed by algae during the return pass. One suitable material is Fluorescent Red Mega 520 (per Sigma Aldrich) which has an absorption peak at 527 nm and an emission peak at 663. Another is DY-480XL (available from Dyomics GmbH), which has an absorption peak of 500 nm and an emission peak at 630 nm.

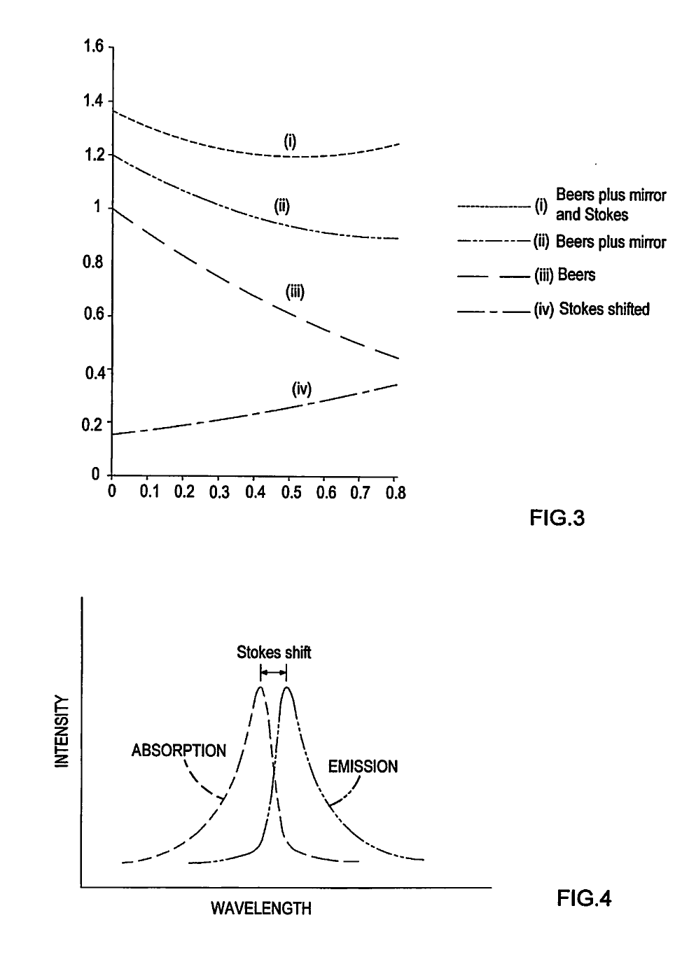

Suitable cheap reflective materials are mirrored acrylic, metallised PET (Mylar) film, white PVC, PET or PMMA sheet). Other thermoplastic material to which reflective or retro-reflective compounds have been applied may also be used. As shown in FIG. 3A incident light L passes through the reactor wall 12 and is absorbed on its first pass through the growth medium 50. Unabsorbed light is reflected at the mirror/reflector 38 and passes back through the growth medium where more energy is absorbed in this second pass (see FIG. 3B). Thus, as illustrated in FIG. 3C, this doubles the effective optical path and therefore working depth of the reactor. Expressed another way, this configuration allows the same absorption efficiency as a conventional single pass to be achieved in a reactor with half the depth.

Thus in the invention bioreactor, canopy shading is reduced because the intensity difference between front and rear is smaller, and thus the average intensity is higher. All algae can acclimate to high light levels, reducing the potential for saturation/photo-inhibition. Furthermore, for the same absorption efficiency as with a single pass, the active mirror configuration results in a 50% reduction in the growth medium volume. Thus the total bioreactor mass, and pump energy requirements, may be reduced. Thus there is less water use, lower bioreactor construction costs and higher net energy ratio (NER) plus simplified downstream procedures.

By sacrificing a small amount of absorption efficiency (i.e. to 80% as compared to ˜90% in an optically dense prior art system) a large reduction in photo-inhibition should be possible, so that photo-inhibition losses of ˜75% can be reduced to less than 20%.

One suitable algae type is Cholorella vulgaris. A growth medium composition is set out below:

Bold Basel Medium with 3-fold nitrogen and vitamins (3N BBM+V)

| Stock Solutions in g/1000 mL in water | | | For 1 L final medium |
|---|---|---|---|
| 1) | 25.0 g | NaNO₂ | 30 mL |
| 2) | 2.5 g | CaCl₂•2H₂O | 10 mL |
| 3) | 7.5 g | MgSO₃•7H₂O | 10 mL |
| 4) | 7.5 g | K₂HPO₄•3H₂O | 10 mL |
| 5) | 17.5 g | KH₂PO₄ | 10 mL |
| 6) | 2.5 g | NaCl | 10 mL |

| | | |
|---|---|---|
| 7) | Trace Element Solution (see below) | 6 mL |
| 8) | Vitamin B₁ (see below) | 1 mL |
| 9) | Vitamin B₁₂ (see below) | 1 mL |

Make up to 1 litre with distilled water. Then autoclave at 15 psi for 15 minutes.

For the trace Element Solution (7) add to 1000 mL of distilled water 0.75 g Na₂EDTA and the minerals in the following sequence:

| | |
|---|---|
| FeCl₂•6H₂O | 97.0 mg |
| MnCl₂•4H₂O | 41.0 mg |
| ZnCl₂•6H₂O | 5.0 mg |
| CoCl₂•6H₂O | 2.0 mg |
| Na₂MoO₄•2H₂O | 4.0 mg |

For Vitamin B₂ (8): use 0.12 g Thiaminhydrochloride in 100 mL distilled water. Filter sterilise.

For Vitamin B₁₂ (9): use 0.1 g Cyanobalamin in 100 mL distilled water, take 1 mL of this solution and add to 99 mL distilled water. Filter sterilise.

A high cell concentration in the growth medium (small chl per cell) is preferred to enable very thin growth medium thickness—of the order of a few mm. This ensures light absorption in a thin layer which overlaps the line foci before the beams can diverge significantly. This also allows replenishment of water in the growth medium following harvesting which dilutes metabolites/catabolites in the medium. The thickness is limited by the need to provide sufficient aqueous volume for dissolved CO₂/O₂ and sufficient thermal mass to prevent dangerous increases in temperature due to solar gain in direct sunlight.

Algal cell concentration N determines the light profile in the media—a high N causes a sharp fall off in light intensity so all the light is absorbed in the PBR. With a low N there is a gentle fall in intensity through the depth of the PBR—i.e. not all of the light is absorbed. Depending upon the PBR reservoir's rear surface modification (e.g. mirroring/disperse reflection or scatter/or fluorescent so as to give a Stokes shift) there will be a concentration N which optimises light absorption/utilisation and therefore maximises growth/yield/reproduction rate.

To hold the system at this optimal concentration (or photostasis) it is preferable to continuously to measure the absorption (i.e. optical density OD) of the algal broth/growth media. This may be effected by a simple LED—detector photometer measuring at the chlorophyll absorption maximum of −675 nm). When the OD exceeds the optimum level a harvest/recharge cycle is triggered. Two identical pumps running off two T sections from the main flow pipe run for a predefined length of time. One removes a set volume of the growth media. The green broth obtained—this is the harvest—can be centrifuged down or otherwise separated to obtain the cells as a solid. The other pump replaces the harvested broth with more nutrient media. The harvest is drawn off upstream of the recharge location. Thus there is no net change in volume—just a reduction of algal concentration from N to Nₒₚₜᵢₘₐₗ.

Naturally, the faster the cells are reproducing, the more frequent the harvest/recharge cycle is required (and the higher the yield). The optimum OD will vary according to species/strain of algae and structure of the PBR.

High levels of oxygen become toxic for all life. In a PBR containing large concentrations of efficiently photosynthesising algae in bright sunlight, large amounts of oxygen will be produced in relatively small liquid volumes, leading to high oxygen concentrations. If this is not effectively removed, the growth medium will become oxygen-saturated and algal growth rate will be limited. The more effective the design of a PBR the higher the photosynthesis efficiency and the greater the O₂ production rate. Effective degassing therefore becomes vital in high efficiency large production systems.

As the present invention generally involves a relatively thin planar configuration reactor, internal sparging to remove oxygen is difficult for reasons of lack of internal volume for bubble formation. Thus in this embodiment the growth medium can be conveyed to an external sparging column. Circulating growth medium flows into the top of a gas contactor tube, down the tube and out of the bottom, whilst air bubbles are fed into the bottom, rise through the liquid and exit through the top. A peristaltic pump or axial pump is used to circulate fluid through the PBR, to the contactor and back to the pump. Alternatively a for a hollow fibre contactor gas contactor can be used (with surface area: volume ratios of the order of 1000 m²/m³). The operation of gas contactors requires growth medium flow in one direction, and gas flow in the other, without significant resistance. Such gas contactors can be used between individual PBR panels to ensure appropriate CO₂ levels and effective O₂ venting, with a common gas flow line to all contactors via a centralised pumping station.

To optimise the intensity profile, precise control of algae concentration enables operation in near photostat mode. This entails using continuous monitoring and regular semi-batch or continuous harvesting (according to photosynthesis rate and biomass production rate, which are dependent on light intensity) to control cell density, hence absorption cross section and total growth medium absorption coefficient and light profile within it, in order to exploit the two pass geometry using a rear reflector (e.g. mirror or diffuse reflector).

For example, for a system in which the alga population doubles every three hours, if harvesting is not performed, one summer's day could see an order of magnitude increase in alga concentration. This results in a transition from optically thin to optically dense system which regulatory changes cannot accommodate and instead acclimatory changes must be made—subject to material costs and downtime losses.

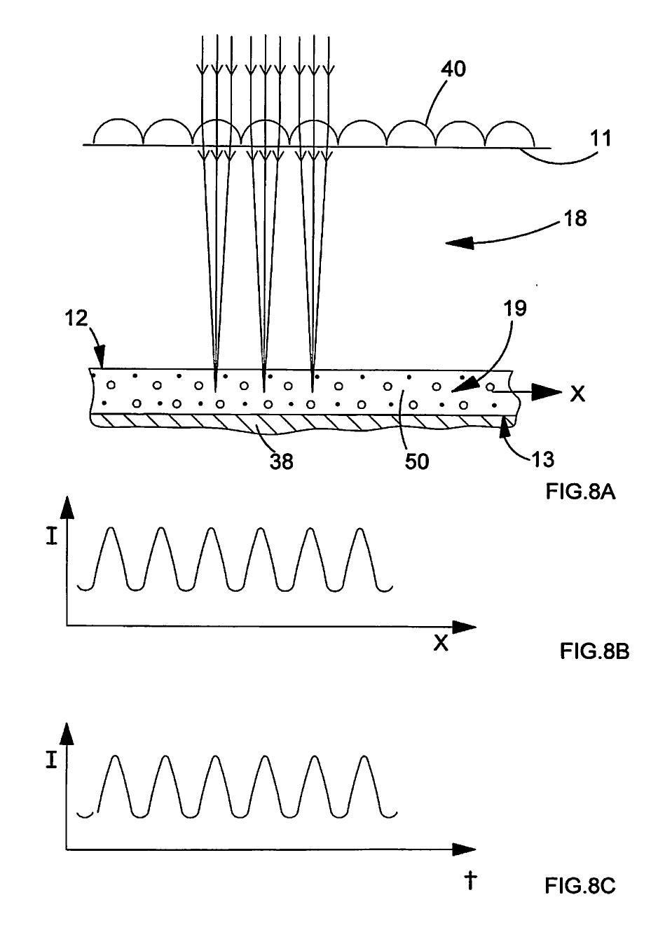

Temporal photo-modulation is provided by mimicking the refractive effect of wavelets (see FIG. 2), though in a more ordered repetitive manner. Specifically, the upper reactor wall 11 is provided with regularly spaced cylindrical (1-D) lensing elements 40, as hereinbefore described. The elements (and associated upper wall) are placed a set distance away from the growth medium in the reactor chamber 19 so as to produce multiple line images of the sun within the growth media. The distance and lens form is selected so that the focal length of the lens element falls in the lower chamber, or nearby so as to concentrate light into relatively light and dark regions. This is illustrated schematically in FIG. 8A.

Such cylindrical lensing elements may be formed by casting, moulding, embossing or by extrusion of plastics sheet incorporating a circular, parabolic or sinusoidal profile, or using Fresnel elements, or holographic films.

The elements are repeated with a small periodicity (e.g. 0.1-1 mm) such that the line foci have a similar periodicity. The spatial photo-modulation of the multiple line foci (FIG. 8B) is transformed into temporal photo-modulation (FIG. 8C) using growth media flow orthogonal to the line images (in direction X)—the alga experience intermittently relatively dark and light zones as they pass through the bright focus and then into the darker ambient light levels between the foci. Thus rather than using canopy shading or a high frequency strobe (as in the prior art) to achieve a flashing light effect, the present invention mimics the action of wavelets to provide high intensity line foci in the growth medium through which the cells pass.

Temporal photomodulation can enhance photosynthesis rates and cultivation of algae. In the present invention the choice of the flow velocity, lens spacing and geometry of lensing features can be selected to give optimum “flash” duration (pulse length) and duty cycle (time between flash) for particular species of alga.

As an example, a lenticular array is composed of parallel one dimensional elongate (linear) lenses (cylindrical lensing elements) with a pitch of 4 lenses per mm (LPMM). This produces multiple line foci spaced at −250 μm. A growth medium flow velocity of 0.1 m/s results in a frequency of 400 Hz (i.e. a 250 μs flash followed by a 2.25 ms “dark” period for a lens arrangement producing a duty cycle of 10:1.

The light intensity may be about 100 to 500 or more microEinsteins. Midday direct sunlight can be about 2000 microEinsteins. Distortions to the image caused by lens aberrations serve to increase the pulse length and reduce the duty cycle. Higher flow velocities may be used to provide higher frequencies, though with increased pump energy requirements.

In overcast conditions with low light levels compared to sunlight, no FLE is required or indeed can be created, and low O₂ levels are produced by the algae. Thus the growth medium flow velocity can be reduced to reduce energy input into the system. Only in direct sun with high intensity is the FLE required to prevent photoinhibition and increase photosynthesis rates. In these circumstances the growth medium flow velocity must be increased, though the energy input is offset by increased productivity as a result of higher light intensities.

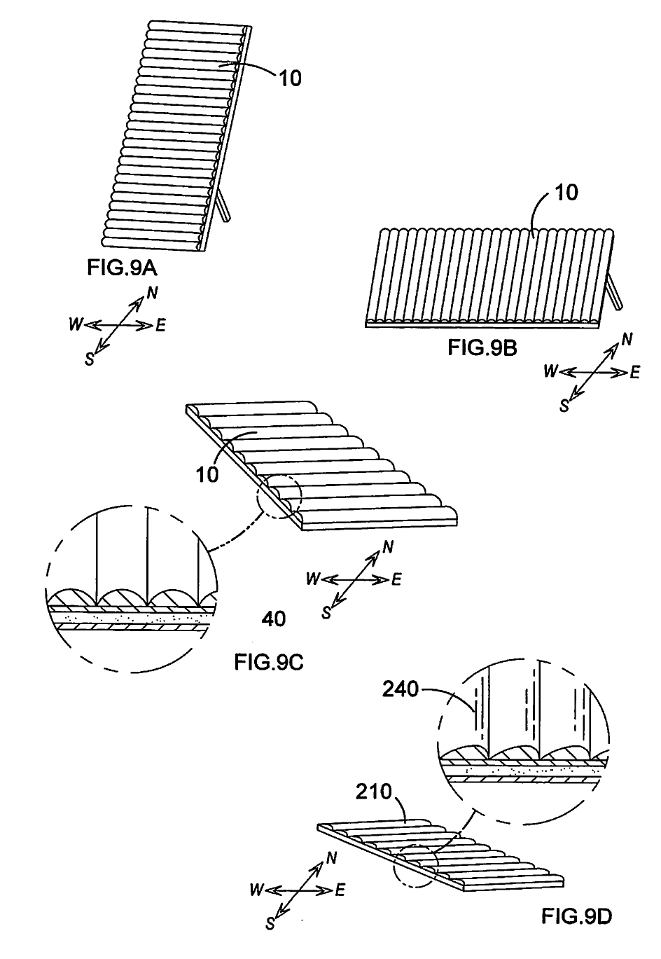

Aberrations in the solar images can cause adjacent foci to blur and even merge. This has the effect of increasing the flash duration, reducing the temporal photomodulation and thus destroying the FLE effect. To minimise aberrations and optimise the FLE, parabolic profile lenses should be used. The passage of the sun across the sky during the day presents problems, though these can be ameliorated with appropriate techniques and four permutations are possible depending on latitude and adoption of tilting geometries. For equatorial regions where the sun is overhead at noon, the PBR can be laid flat on the ground with the lenses aligned East-West and growth medium flow North-South (or S—N), as shown in FIG. 9C. Movement of the sun from E to W during the day is therefore perfectly accommodated by the E-W oriented 1-D cylinder lenses, resulting in few distortions. Moreover, at midday when solar intensity is maximum, the rays are perfectly on axis and aberrations are minimal—the ideal situation. The best solar images are produced in equatorial regions at noon, resulting in optimum FLE.

Tilting of the PBR 10 with respect to the sun (FIGS. 9A and 9B) can be performed to reduce aberrations due to solar elevation so that solar rays are near-normal to the upper planar surface of the PBR and therefore predominantly on-axis. Two geometries are possible for tilting PBRs with flow horizontally (FIG. 9B) or vertically (FIG. 9A).

FIG. 9D shows a third embodiment of a reactor 210 which has asymmetric (lop-sided) section elongate lens elements 240, for which see further details below.

Reactor tilt may be controlled to ensure that incident light approaches the reactor perpendicular, or substantially perpendicular, to the reactor surface. Thus the angle of tilt may be fixed at an optimum value for the latitude, or varied continuously, or stepwise, throughout the day and from season to season, using an automated mechanism to track sun elevation. Tilting ensures solar elevation can be accommodated to minimise aberrations, reduces reflection losses and can increase total annual solar energy collection per square metre of PBR deployed. Additionally, with a tilting arrangement the front and rear surfaces are available for heat loss to the air.

Alternatively, for a simpler recumbent system in high latitude regions, with lenses aligned W-E and growth medium flow N-S, the lens arrays are aligned off-axis at an angle approximately equal to the average solar elevation, so as to reduce aberrations due to variations in solar elevation, minimise size of the focus and thereby optimise photomodulation

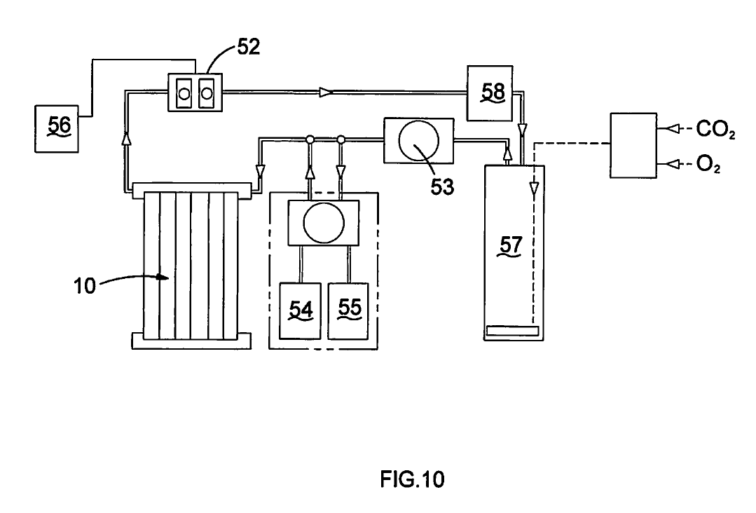

The ancillary bioreactor system services are schematically illustrated in FIG. 10. A PBR 10 is provided mounted on a substrate (not shown). Growth medium is continuously circulated (culture flow) through PBR reservoir chambers (not shown) by a pump station 53. A harvest station 54 and growth medium recharge station 55 communicate with the growth medium flow line so as to permit continuous or batchwise removal of algae (dilution of the growth medium). Following removal, harvesting may be performed by conventional methods such as settlement, floatation or filtration. A monitoring & control station 56 receives signals from in-line sensors 52. These sensors sample carbon dioxide and oxygen levels and algae absorption/concentration, and transmit data for analysis in the control station. This can then be used to adjust algal concentration by harvesting growth media from the system and recharging with nutrient media. Temperature and solar data (intensity) may be sensed from the atmosphere and target algal concentration adjusted accordingly. A temperature control unit 58 can be used to adjust the temperature of the growth medium. A sparger column 57 is placed in the flow path into each PBR. The sparger removes excessive oxygen from, and adds carbon dioxide to the growth medium as required to ensure efficient photosynthesis.

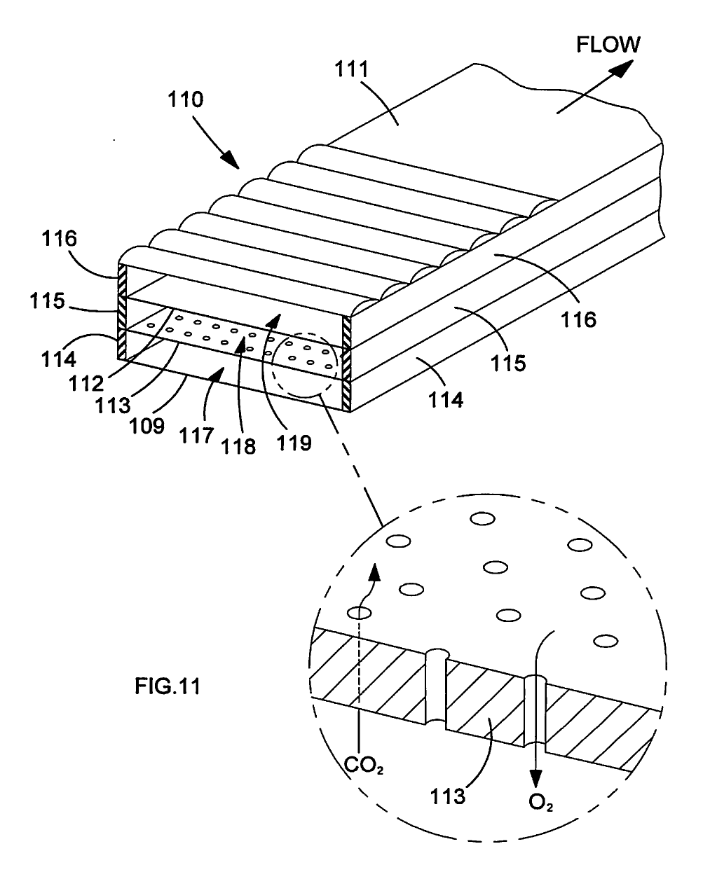

In FIG. 11 a second embodiment elongate photo-bioreactor (not to scale) for photosynthesis of algae is shown generally as 110. The reactor is rectilinear and planar in configuration. The reactor has an upper planar wall 111, an intermediate planar wall 112, a planar base wall 113 and a bottom wall 109. Upstanding orthogonal sidewalls 114, 115, 116 are provided at each side of the reactor and internal side walls/sub-dividing walls 120. Sidewall 114 extends from an upper side of the side region of the bottom wall to a lower side of a side region of the base wall. Similarly sidewall 115 extends from the base wall to the intermediate wall. Sidewall 116 extends from the intermediate wall to the upper wall. The planar walls are each 50-1000 microns thickness and are preferably each sufficiently flexible to be elastically rolled to a large-radius spool.

The sidewalls are formed of closed-cell foam material which is impermeable to aqueous media and flexible, or of similar material to the planar walls and are bonded to the planar walls. The planar bottom, base, intermediate and upper layers are thin and made of resilient clear plastics sheet material. Thus the reactor may be stored and transported coiled onto a large-radius spool (not shown) by virtue of the flexibility of the structure.

Together with the sidewalls, the planar walls define three internal compartments 117, 118 and 119. A middle compartment 118 serves as a reservoir for growth medium. A lower compartment 117 is a gas exchange compartment (as will be described hereinafter) and the upper compartment 119 is a heat exchange layer.

The proximal and distal end caps (not shown) are attached to proximal and distal end regions of the reactor to close the reactor. The end caps are formed with inlet and outlet ports (not shown) which charge and discharge fluid into/from the compartments.

These ports allow the fluid (gas or liquid) in the upper compartment 119 to be changed according to requirements for temperature regulation: in cold climes/at night, low thermal conductivity fluids can be used (air or argon) to insulate the reactor whereas in hot climates or during high solar gain due to intense sunlight, high thermal conductivity fluids are used (Helium or water) to increase heat transfer from the growth medium so as to prevent the growth medium reaching excessive temperatures.

The intermediate wall 112 is formed of clear thermoplastics sheet material. The base wall 113 is formed of microporous membrane material. The air-filled pores cause light to be reflected so that the sheet is highly reflective, typically about 98%. The microporous membrane provides for gas exchange i.e. O₂ out and CO₂ in. Compartment 117 is provided below the PBR's reservoir and is separated from the reservoir's growth medium in this embodiment by microporous polypropylene. The microporous polypropylene has a pore size of approximately 0.4 microns with specified air flow rate of −16 Lpm/3.7 cm²@0.9 bar/MVTR˜1500 g/m²/24 hr).

The growth medium circulates on the reservoir side of the membrane in one direction, while air with enhanced CO₂ concentration levels (about 5%) flows in the opposite direction on the other side of the membrane (i.e. counter-current). Existing control techniques such as gas flow through contactor and computer control of nutrient levels, pH etc. may be used.

The point at which a hydrophobic microporous membrane ceases to be waterproof, the water breakthrough pressure, is determined by pore size and is inversely proportional to air flow rate across the membrane. A compromise must be found and a typical hydrophobic microporous membrane with 0.4 micron pores is can withstand pressures of up to about 10 psi or a 6 m water column. Below this pressure water is contained though gases may diffuse across if there is a concentration gradient. Thus, the pores are impermeable to growth medium (water) but allow gas transfer between the growth medium reservoir 118 and the lower compartment 119. In use photosynthesising algae will deplete the growth medium of carbon dioxide and generate oxygen thereby setting up the concentration gradient (these will be large if the growth medium layer is thin). Thus the lower compartment contains air or elevated CO₂ levels as a source of carbon dioxide and into which oxygen can diffuse so as to prevent growth inhibition of the growth medium by excessive oxygen content. To enhance the gas exchange process the gas in the lower compartment may flow in a counter-current direction with respect to the growth medium flow in the compartment above. The gas in the lower compartment is preferably air, optionally air having elevated CO₂ levels (relative to ambient air).

The upper wall 111 is formed of clear thermoplastic sheet material. An upper surface of the upper wall is formed with a plurality of parallel elongate transverse lenses 140 (only the first seven shown for clarity. The lenses each have a uniform section (i.e. are 1-dimenional) and have an essentially parabolic or spherical section which represents a minor chordal portion of a circle. The lenses may be applied for example by embossing using a rolling mill. Alternatively an array of Fresnel or holographic lenses may be used. The use of a thin film and flexible side walls allows the PBR to be coiled along the longitudinal axis.

The lenses provide linear foci which fall in or (just above or just below) the growth medium. The position of the foci will depend upon whether a gas or liquid is present in the upper compartment. The key requirement is that there be generated regions of relatively high intensity (i.e. higher than the incident intensity) and regions of relatively low light intensity. Thus with air in the upper compartment the foci will be in front of the growth medium. With water in the upper compartment the foci will be behind the growth medium. Nevertheless there will still be formed pronounced bright lines in the growth medium which ensure photomodulation takes place, though not as pronounced as at the focus. For example, the light intensity spatial distribution (measured in the direction of lens width) changes from 5% light 95% dark with an optimum focus (duty cycle 1:20) to 10% light 90% dark (duty cycle 1:10) when the focus is just outside the growth medium. To achieve the benefits of the invention a spatial intensity distribution ratio of up to 50% light 50% dark is sufficient to provide a flashing light effect. The lenses may be provided in a pitch of about 4 lenses per mm of reactor length, although other densities may be used according to requirements.

The growth medium is continuously moved through the growth medium chamber so that a flashing light effect acts upon the growth medium as the algae travel into and out of light and relatively dark regions. Liquid deflecting or mixing features such as surface striations (not shown) in an underside of the intermediate wall aid in mixing of the growth medium as it flows past.

In all embodiments the concentration of algae in the growth medium should be controlled to ensure that the appropriate amount of light makes it to the rear mirror (i.e. it operates in photostat mode). If the algae is, or becomes over time, too optically dense then significant amounts of light will not penetrate to the reflective rear reservoir surface and the benefits of bending Beers law (as previously described) will not be realised. Thus the growth medium requires periodic or continuous harvesting of algae by removing some of the growth medium and replacing it with nutrient medium without cells. This harvested growth medium is subsequently processed to derive the desired products.

In all specific embodiments, and indeed in accordance with the general invention per se, the reactor compartments for growth medium, heat exchange gas or gas exchange may each be subdivided. For example each compartment may be subdivided into elongate side by side channels (note shown in the figures). The channels' sidewalls may enhance structural rigidity. A fluid pressure within the channels may be maintained at above ambient pressure so as to maintain the reactor rigidity. The channels may be individually fed with fluid (such as growth medium, heat exchange gas or liquid or gas exchange medium) or may be fed by a manifold for each compartment which distributes fluid into all, or a selected multiple of channels.

A third embodiment is identical to the first or second embodiments, with the exception of the lens configuration. The configuration is shown in FIG. 9D. In this embodiment a reactor 210 is provided with a lens array made up of parallel elongate lenses 240. The lenses have an asymmetric cross section, taking the form of a lop-sided spheric. The lens elements correct for the oblique (off normal axis) fall of light at low sun elevations. Thus these lenses are suitable for use in medium to high latitudes. Thus light falling on the lenses at an oblique angle (corresponding to high latitude reactor sites) suffers minimal aberrations and emerges along a normal axis substantially perpendicular to the plane of the reactor to form parallel foci.

The protection scope of the present invention is set forth in the annexed Claims and cannot be restricted to the illustrative disclosures given above, under the detailed description. It is because a person skilled in the relevant art can obviously produce similar embodiments under the light of the foregoing disclosures, without departing from the main principles of the present invention.

What is claimed is:

1. A method of cultivating biomass by photosynthesis comprising providing a fluid growth medium in which biomass is dispersed and which light can penetrate, exposing the growth medium to a source of incident light characterised in that the incident light is lensed using lensing means so as to form one or more elongate light foci in the growth medium, and transporting growth medium through said foci in a direction transverse or oblique with respect to a longitudinal foci axis so as to provide temporal photo-modulation in the growth medium.

2. The method of claim 1 wherein the frequency of the temporal photo-modulation is controlled by adjusting a growth medium flow velocity.

3. The method of claim 1 wherein the growth medium velocity is less than 0.3 m/s.

4. The method of claim 1 wherein the temporal photo-modulation frequency is from 100 to 1000 Hz.

5. The method of claim 1 wherein a photo-modulation duty cycle is set by selection lens spacing or lens focal length in the lensing means, or proximity of the growth medium to a lens focus.

6. The method of claim 1 wherein there are plural light foci adapted to produce a photo-modulation duty cycle (low light to high light) of at least 2:1.

7. The method of claim 1 wherein there are plural light foci adapted to produce a photo-modulation duty cycle (low light to high light) of 10:1 or more.

8. The method of claim 1 wherein a light-reflecting surface is disposed beneath the growth medium so as to reflect at least a portion of any incident light which has passed unabsorbed through the growth medium back through the growth medium.

9. A photo-bioreactor for cultivating biomass by photosynthesis comprising:
(a) a generally planar reservoir for containing a fluid growth medium while allowing incident light to penetrate the growth medium and
(b) a plurality of fixed lensing means distributed in an array above and generally parallel to the planar reservoir, each lensing means being adapted to concentrate light passing therethrough into the growth medium so as to form a correspondingly distributed plurality of regions of relatively high light intensity and relatively low light intensity in the growth medium, and
wherein fluid transport means are provided for inducing a flow of growth medium along the reservoir so that the growth medium passes sequential regions of relatively high and relatively low intensity light so as to produce temporal photo-modulation in the growth medium.

10. The photo-bioreactor of claim 9 wherein each lensing means is adapted to form an associated discrete focus in the growth medium and/or at a location adjacent the growth medium which is sufficiently close to concentrate light within the growth medium so as to form relatively light and relatively dark regions in the growth medium

11. The photo-bioreactor of claim 9 wherein the lensing means in the array are configured and arranged to provide an area and distribution of high intensity light regions within the reservoir which provides for growth medium flowing through the reservoir a duty cycle (low light to high light) of at least 2:1.

12. The photo-bioreactor of claim 9 wherein the lensing means in the array are configured and arranged to provide an area and distribution of high intensity light regions within the reservoir which provides for growth medium flowing through the reservoir a duty cycle (low light to high light) of 10:1 or more.

13. The photo-bioreactor of claim 9 wherein the lensing means are distributed in a generally planar array.

14. The photo-bioreactor of claim 9 wherein each lensing means is adapted to form an elongate focus.

15. The photo-bioreactor of claim 9 wherein the plurality of fixed lensing means comprises an array of convergent lenses through which incident light passes before passing into the growth medium, wherein the lens array is adapted to focus incident light into a plurality of foci located in the growth medium.

16. The photo-bioreactor of claim 9 wherein the lensing means comprises a plurality of elongate lenses which each provide a linear focus within the growth medium.

17. The photo-bioreactor of claim 16 wherein the elongate lenses are arranged in a generally parallel and/or side-by-side orientation, and preferably substantially co-planar with one another.

18. The photo-bioreactor of claim 9 wherein the focal length of each lensing means is between 0.5 mm and 100 mm.

19. The photo-bioreactor of claim 9 wherein the lensing means array is provided in or on a clear sheet material.

20. The photo-bioreactor of claim 9 wherein the lensing means array is provided on or in a top wall of the reactor.

21. The photo-bioreactor of claim 9 wherein each lensing means in the array comprises an aspheric surface and is preferably parabolic in form.

22. The photo-bioreactor of claim 9 wherein the lensing means array is generally planar, spaced apart from the growth medium and located before the growth medium in the incident light path.

23. The photo-bioreactor of claim 9, comprising a light-reflecting surface which is disposed beneath the growth medium so as to reflect at least a portion of any incident light which has passed unabsorbed through the growth medium back through the growth medium.
